(19) United States
(12) Patent Application Publication
(10) Pub. No.: US 2015/0197778 A1
(43) Pub. Date: Jul. 16, 2015

(54) TRIGLYCERIDE PRODUCTION IN E. COLI

(71) Applicant: Tuffs University, Medford, MA (US)

(72) Inventors: Kyongbum Lee, Winchester, MA (US); Joanna Rucker, Somerville, MA (US); Julie Paul, Somerville, MA (US); Blaine Pfeifer, Somerville, MA (US)

(21) Appl. No.: 14/391,582

(22) PCT Filed: Apr. 11, 2013
(86) PCT No.: PCT/US2013/036131

§ 371 Date: Oct. 9, 2014

Related U.S. Application Data
(60) Provisional application No. 61/622,781, filed on Apr. 11, 2012.

(51) Int. Cl. C12P 7/64; C12N 9/10; C12N 9/16

(52) U.S. Cl. CPC C12P 7/6463; C12N 9/16; C12N 9/1029; C12Y 203/0102; C12Y 301/03004

(57) ABSTRACT

A recombinant E. coli cell including nucleic acid sequences that encode phosphatidic acid phosphatase (PGP) and diacylglycerol acyltransferase (DGAT). Upon expression of the PGP and DGAT, the cell produces a triglyceride having a C16 acyl chain. Also provided is a method for producing triglycerides by culturing the recombinant E. coli cell.

### CROSS REFERENCE TO RELATED APPLICATIONS

This application claims priority to U.S. Provisional Application No. 61/622,781, filed Apr. 11, 2012, the contents of which are hereby incorporated in their entirety.

### BACKGROUND

Triglycerides have a variety of uses, including as a raw material for producing biodiesel and as a component of a variety of household products, including lotions, lubricants and cooking oils. They are typically sourced from plant oils, such as soybean and sunflower oil.

Triglycerides are used to produce biodiesel through esterification of the fatty acids in the triglycerides with an alcohol such as methanol or ethanol.

Triglyceride accumulation has been observed in bacteria of the Actinomycetes genera. These bacteria include Mycobacterium, Nocardia, Rhodococcus, and Streptomyces. A variety of neutral lipids have been identified in these bacteria, including polyhydroxyalkanoic acids, wax esters, and triglycerides.

E. coli has been used for the production of a variety of compounds on a commercial scale, including therapeutic proteins and small molecules in the pharmaceutical industry, and for commodity chemical production, such as ethanol for the biofuel industry.

There is a need to develop an efficient method for producing triglycerides in E. coli.

### SUMMARY

This invention relates to the production of triglycerides in a bacterial system.

In one aspect, a recombinant E. coli cell is disclosed comprising nucleic acid sequences that encode phosphatidic acid phosphatase (PAP, also known as PGP) and diacylglycerol acyltransferase (DGAT), wherein the cell produces triglycerides having a C16 acyl chain.

In another aspect, a method for producing triglycerides is provided. The method includes the steps of providing a recombinant E. coli cell that expresses PGP and DGAT, culturing the cell in a media, and isolating the triglycerides from the media.

Another aspect of the invention is a liquid chromatography-mass spectrometry (LCMS) method for analyzing the production of triglycerides by engineered E. coli strains.

The details of one or more embodiments of the invention are set forth in the description below. Other features, objects, and advantages of the invention will be apparent from the description and from the claims. All references cited herein are hereby incorporated by reference in their entirety.

### DETAILED DESCRIPTION

A recombinant E. coli cell is provided, containing nucleic acid sequences that encode phosphatidic acid phosphatase (PGP) and diacylglycerol acyltransferase (DGAT). The recombinant cell upon expression of the PGP and DGAT, produces triglycerides having at least one C16 acyl chain. The triglycerides that can be produced include but are not limited to 16:0/16:0/16:1, 16:0/16:0/18:1, 18:1/16:0/16, 18:1/18:1/16:0, 16:0/16:1/16:1, 16:0/16:0/14:0, 16:0/16:0/16:0, 18:1/18:1/18:1, and 18:1/16:1/14:0.

The DGAT can be Acinetobacter spp., Mycobacteria spp. (e.g., M. tuberculosis, M. smegmatis, M. bovis, M avium, M. leprae), or Streptomyces coelicolor DGAT. In one embodiment, the DGAT is Acinetobacter spp. DGAT. In a preferred embodiment, the DGAT is A. calcoaceticus DGAT having the amino acid sequence of SEQ ID NO: 1:

```
A. calcoaceticus DGAT (Genbank Accession number: AAO17391)
(SEQ ID NO: 1)
    1 MRPLHPIDFI FLSLEKRQQP MHVGGLFLFQ IPDNAPDTFI QDLVNDIRIS KSIPVPPFNN
  61 KLNGLFWDED EEFDLDHHFR HIALPHPGRI RELLIYISQE HSTLLDRAKP LWTCNIIEGI
121 EGNRFAMYFK IHHAMVDGVA GMRLIEKSLS HDVTEKSIVP PWCVEGKRAK RLREPKTGKI
181 KKIMSGIKSQ LQATPTVIQE LSQTVFKDIG RNPDHVSSFQ APCSILNQRV SSSRRFAAQS
241 FDLDRFRNIA KSLNVTINDV VLAVCSGALR AYLMSHNSLP SKPLIAMVPA SIRNDDSDVS
301 NRITMILANL ATHKDDPLQR LEIIRRSVQN SKQRFKRMTS DQILNYSAVV YGPAGLNIIS
361 GMMPKRQAFN LVISNVPGPR EPLYWNGAKL DALYPASIVL DGQALNITMT SYLDKLEVGL
421 IACRNALPRM QNLLTHLEEE IQLFEGVIAK QEDIKTAN
```

Not to be bound by theory, a highly conserved motif (HHXXXDG) corresponding to amino acids 132-138 of A. calcoaceticus DGAT may be a catalytic site responsible for ester bond formation. Thus, DGAT enzymes from organisms other than Acinetobacter can be used in the invention described herein.

Additionally, the recombinant E. coil cell can express a PGP having the amino acid sequence of SEQ ID NO: 2:

```
E. coli PGP B (also known as PAP) (Genbank Accession number: AAB36618)
(SEQ ID NO: 2)
    1 MRSIARRTAV GAALLLVMPV AVWISGWRWQ PGEQSWLLKA AFWVTETVTQ PWGVITHLIL
  61 FGWFLWCLRF RIKAAFVLFA ILAAAILVGQ GVKSWIKDKV QEPRPFVIWL EKTHHIPVDE
121 FYTLKRAERG NLVKEQLAEE KNIPQYLRSH WQKETGFAFP SGHTMFAASW ALLAVGLLWP
181 RRRTLTIAIL LVWATGVMGS RLLLGMHWPR DLVVATLISW ALVAVATWLA QRICGPLTPP
241 AEENREIAQR EQES
```

The recombinant E. coli can contain an expression vector that includes the nucleic acid sequences described above. In one embodiment, the recombinant E. coli contains one expression vector including a nucleic acid encoding both PGP and DGAT. In another embodiment, the recombinant E. coli contains two expression vectors, one including a nucleic acid encoding PGP and one including a nucleic acid encoding DGAT.

As mentioned above, a method for producing triglycerides is provided. In the method, a recombinant E. coli cell that expresses PGP and DGAT is obtained. The cell is cultured in a media allowing for production of triglycerides, and the triglycerides are isolated from the media.

The DGAT can be Acinetobacter spp. DGAT. The DGAT can have the amino acid sequence of SEQ ID NO: 1. The PGP can have the amino acid sequence of SEQ ID NO: 2. Additionally, the media can contain at least 5 g/L glucose.

Culturing the E. coli cell that expresses PGP and DGAT in LB medium containing 5 g/L glucose for eight hours can yield 0.7-1.1 mg/L triglycerides, corresponding to a maximum of 2 g/L dry cell weight (DCW).

In another embodiment, E. coli expressing PGP and DGAT can also express phasin. Phasin is a lipid binding protein that can coat lipid droplets to protect them from degradation and can act as a point of seeding for triglyceride formation.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

Expression of DGAT and PGP in E. coli

A plasmid containing the DGAT gene (Genbank Accession No.: AAO17391; SEQ ID NO: 1) and the PGP gene (Genbank Accession No.: AAB36618; SEQ ID NO: 2) was introduced into E. coli strain EDE3. The resulting recombinant strain was designated EDE3(DGAT-PGPB).

A culture of EDE3(DGAT-PGPB) was grown in LB media at 37° C. to an optical density (OD) of 0.5-0.7. Isopropylthiogalactopyranoside (IPTG) was added to the culture to induce expression of PGP and DGAT. The induced culture was further incubated at 37° C.

A 1 mL sample was removed from the culture at 1 h, 2 h, 4 h, and 24 h after IPTG addition. The samples were analyzed by sodium dodecylsulfate polyacrylamide gel electrophoresis (SDS-PAGE) to detect expression of the PGP and DGAT proteins. SDS-PAGE analysis was performed using standard methods. Results indicated that expression of PGP and DGAT was weak at 1 hour and strong at 2 hours and 4 hours. Expression of DGAT was reduced after 8 hours, and expression of PGP was still observed at 24 hours.

Enzyme activity of DGAT produced by recombinant E. coli as described above was compared to that of DGAT produced by A. calcoaceticus strain ADP1. The DGAT enzyme produced by the recombinant E. coli demonstrated the same reaction rates as native A. calcoaceticus DGAT. DGAT enzyme activity was 7.96 pmol (mg min)-1 in the presence of 3.75 mM 1,2-diaplmitoyl-rac-glycerol and [1-¹⁴C] palmitoyl-CoA. The DGAT demOnstrated a Km of 21.1 μM and a Vmax of 54.3 pmol(mg min)-1 using palmitoyl-CoA as a substrate.

Liquid Chromatography/Mass Spectrometry

A LCMS method was developed to analyze triglyceride production of E. coli strain EDE3(DGAT-PGPB) described above.

A 30 ml culture of EDE3(DGAT-PGPB) in LB media containing 5 g/L glucose was grown at 37° C., to an OD of 0.5-0.7. The culture was induced with IPTG and incubated again at 37° C. A 1 mL sample was taken from the culture at 2 h, 4 h, 8 h, and 24 h after IPTG addition. Samples were centrifuged to pellet the cells and the media aspirated.

Lipids were extracted from cell samples by adding 800 μL of 2:1 chloroform/methanol (v/v) to the cell pellet and vortexing. 200 μL of water was added and the samples were centrifuged to separate the solvents into two phases. 500 μL of the total 600 μL lower phase was collected, ensuring not to collect any of the interface. The collected lower phase was placed in a new tube stored at −20° C.

For LCMS analysis, 50 μL of the lower phase was evaporated to dryness and 2 ml of isopropyl alcohol (IPA) was added. The samples were kept at 4° C.

LCMS analysis was performed using (i) an Agilent 1200 Series (degasser, bin pump, FC/ALS Therm, HiP-ALS), (ii) an Agilent 1100 Series (column oven), and (iii) an Applied Biosystems 3200 Q Trap (MDS SCIEX - MS/MS) using the instrument settings below.

Source/Gas settings: Ion Source: Turbo Spray, Curtain Gas=10.0, Collision Gas=Medium, IonSpray Voltage=5000.0, Temperature=200.0, Ion Source Gas 1=50.0, Ion Source Gas 2=55.0, Interface Heater=On. Compound settings: Declustering Potential=70.0, Entrance Potential=10.0, Collision Energy=50.0, Collision Cell Exit Potential=2.5, Scan Type—MRM, Polarity—Positive.

First, 10 μL of each sample was loaded onto an Ascentis Express C18 15 cm×2.1 mm, 2.7 μm column. The column oven temperature was set to 55° C.

Mobile phase A contained acetonitrile and mobile phase B contained IPA, 10 mM ammonium formate, and 0.1% formic acid. The column was equilibrated for 6.0 minutes with 50% mobile phase B, increased to 60% mobile phase B at 7.0 minutes, increased to 80% mobile phase B at 11.0 minutes, increased to 100% mobile phase B at 14.0 minutes and decreased to 50% mobile phase B at 15.0 minutes. The flow rate was 0.25 ml/min.

The mass scan points are listed in Table 1 below. The Q1 mass is the mass of the triglyceride plus a molecule of NH₄⁺. The Q1 masses were as listed on the Scripps Center for Metabolomics database (found on the World Wide Web at address metlin.scripps.edu/metabo_search_alt2.php). Q3 masses were calculated as the mass of the triglyceride minus the mass of a fatty acid chain in the triglyceride, including the oxygen.

TABLE 1
Masses of triglycerides and triglyceride fragments in cell extracts

| Q1 Mass (Da) | Q3 Mass (Da) | Time (msec) | ID |
|---|---|---|---|
| 796.700 | 523.500 | 100.0 | 16:0/14:0/16:0-16:0* |
| 796.700 | 551.500 | 100.0 | 16:0/14:0/16:0-14:0* |
| 820.700 | 549.500 | 100.0 | 16:1/16:1/16:0-16:1* |
| 820.700 | 547.500 | 100.0 | 16:1/16:1/16:0-16:0* |
| 820.700 | 521.500 | 100.0 | 18:1/14:0/16:1-18:1* |
| 822.800 | 549.500 | 100.0 | 16:0/16:0/16:1-16:0* |
| 822.800 | 551.500 | 100.0 | 16:0/16:0/16:1-16:1* |
| 824.800 | 551.800 | 100.0 | 16:0/16:0/16:0-16:0* |
| 848.800 | 549.500 | 100.0 | 18:1/16:0/16:1-18:1* |
| 848.800 | 575.500 | 100.0 | 18:1/16:0/16:1-16:0* |
| 848.800 | 577.500 | 100.0 | 18:1/16:0/16:1-16:1* |
| 850.800 | 551.500 | 100.0 | 16:0/16:0/18:1-18:1* |
| 850.800 | 577.500 | 100.0 | 16:0/16:0/18:1-16:0* |
| 876.800 | 577.500 | 100.0 | 18:1/18:1/16:0-18:1* |
| 876.800 | 603.500 | 100.0 | 18:1/18:1/16:0-16:0* |
| 902.800 | 603.500 | 100.0 | 18:1/18:1/18:1-18:1* |

Recombinant E. coli engineered to express both PGP and DGAT, i.e., strain EDE3(DGAT-PGPB), primarily produced the triglyceride 16:0/16:0/16:1 as well as secondary constituents 16:0/16:0/18:1 and 18:1/16:0/16:1. A summary of the triglycerides produced are shown in Table 2. No triglyceride production was observed in the parental E. coli strain EDE3.

TABLE 2
Triglyceride composition (mol %) of
E. coli strain EDE3(DGAT-PGPB)

| Triglyceride species | % Total triglycerides |
|---|---|
| 16:0/16:0/16:1 | 32.7% |
| 16:0/16:0/18:1 | 20.8% |
| 18:1/16:0/16:1 | 19.8% |
| 18:1/18:1/16:0 | 11.1% |
| 16:0/16:1/16:1 | 7.6% |
| 16:0/16:0/14:0 | 3.2% |
| 16:0/16:0/16:0 | 2.7% |
| 18:1/18:1/18:1 | 1.8% |
| 18:1/16:1/14:0 | 0.3% |

The following references each include information that can be used to better understand the background of this invention: Kalscheuer, Rainer and Steinbuchel, Alexander. Mar. 7, 2003, The Journal of Biological Chemistry, pp. 8075-8082; Icho, Tateo. November 1988, Journal of Bacteriology, pp. 5117-5124; Icho, Tateo and Raetz, Christian R. H. 1983, Journal of Bacteriology, pp. 722-730; Dillon, Deirdre A., et al. 1996, The Journal of Biological Chemistry, pp. 30548-30553; Kalscheuer, Rainer, et al. s.l. : Applied and Environmental Microbiology, February 2006, pp. 1373-1379; Santala, Suvi, et al. 36, s.l. : Microbial Cell Factories, 2011, Vol. 10; Kalscheuer, R., Stolting, T. and Steinbuchel, A. s.l. : Microbiology, 2006, Microbiology, Vol. 152(Pt9), pp. 2529-36; Steen, Eric J, et al. s.l. : Nature, 2010, Vol. 463; Serrano-Vega, Maria Josefa, et al. s.l. : Journal of Chromatography, 2003, Vol. 786, pp. 221-228; Alvarez, Adrian F., et al. s.l. : Microbiology, 2008, Vol. 154, pp. 2327-2335; Dani, K. G. Srikanta, et al. s.l. : Plant Biology, 2010; Bokinsky, Gregory, et al. 50, PNAS, 2011, Vol. 108, pp. 19949-19954; Daniel, jaiyanth, et al. 15, s.l. : Journal of Bacteriology, August 2004, Vol. 186, pp. 5017-5030; Hernandez, Martin, et al. 600, s.l. : BMC Genomics, 2008, Vol. 9; Sirakova, Tatiana D., et al. s.l. : Microbiology, 2006, Vol. 152, pp. 2717-2725; Sorger, D and Daum, G. s.l. : Applied Microbiol Biotechnology, 2003, Vol. 61, pp. 289-299; Duan, Yangkai, et al. 5, s.l. : PLoS ONE, 2011, Vol. 6; and Yen, Chi-Liang Eric, et al. s.l. : Journal of Lipid Research, 2008, Vol. 49.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A recombinant E. coil cell, comprising nucleic acid sequences that encode phosphatidic acid phosphatase (PGP) and diacylglycerol acyltransferase (DGAT), wherein, upon expression of the PGP and DGAT, the cell produces a triglyceride having a C16 acyl chain.

2. The recombinant E. coli cell of claim 1, wherein the DGAT is Acinetobacter spp. DGAT.

3. The recombinant E. coli cell of claim 2, wherein the DGAT has the amino acid sequence of SEQ ID NO: 1.

4. The recombinant E. coli cell of claim 1, wherein the PGP has the amino acid sequence of SEQ ID NO: 2.

5. The recombinant E. coli cell of claim 3, wherein the PGP has the amino acid sequence of SEQ ID NO: 2.

6. The recombinant E. coli of claim 1, wherein the nucleic acid sequences are contained in an expression vector.

7. The recombinant E. coli of claim 1, wherein the triglyceride has a C18 acyl chain.

8. The recombinant E. coli of claim 1, wherein the triglyceride has a C14 acyl chain.

9. The recombinant E. coli of claim 1, wherein the triglyceride has at least two C16 acyl chains.

10. The recombinant E. coli of claim 5, wherein the triglyceride has a C18 acyl chain.

11. The recombinant E. coli of claim 5, wherein the triglyceride has a C14 acyl chain.

12. The recombinant E. coli of claim 5, wherein the triglyceride has at least two C16 acyl chains.

13. The recombinant E. coli of claim 1, wherein the cell produces at least 2 g/L triglycerides based on dry cell weight.

14. The recombinant E. coli of claim 5, wherein the cell produces at least 2 g/L triglycerides based on dry cell weight.

15. A method for producing triglycerides, comprising providing a recombinant E. coli cell that expresses PGP and DGAT, culturing the cell in a media to produce triglycerides, and isolating the triglycerides from the media.

16. The method of claim 15, wherein the DGAT is Acinetobacter spp. DGAT.

17. The method of claim 16, wherein the DGAT has the amino acid sequence of SEQ ID NO: 1.

18. The method of claim 15, wherein the PGP has the amino acid sequence of SEQ ID NO: 2.

19. The method of claim 17, wherein the PGP has the amino acid sequence of SEQ ID NO: 2.

20. The method of claim 15, wherein the media contains at least 5 g/L glucose.


SEQUENCE LISTING (2 sequences)

SEQ ID NO: 1 - protein, 458 aa, Acinetobacter calcoaceticus
MRPLHPIDFIFLSLEKRQQPMHVGGLFLFQIPDNAPDTFIQDLVNDIRISKSIPVPPFNNKLNGLFWDEDEEFDLDHHFRHIALPHPGRIRELLIYISQEHSTLLDRAKPLWTCNIIEGIEGNRFAMYFKIHHAMVDGVAGMRLIEKSLSHDVTEKSIVPPWCVEGKRAKRLREPKTGKIKKIMSGIKSQLQATPTVIQELSQTVFKDIGRNPDHVSSFQAPCSILNQRVSSSRRFAAQSFDLDRFRNIAKSLNVTINDVVLAVCSGALRAYLMSHNSLPSKPLIAMVPASIRNDDSDVSNRITMILANLATHKDDPLQRLEIIRRSVQNSKQRFKRMTSDQILNYSAVVYGPAGLNIISGMMPKRQAFNLVISNVPGPREPLYWNGAKLDALYPASIVLDGQALNITMTSYLDKLEVGLIACRNALPRMQNLLTHLEEEIQLFEGVIAKQEDIKTAN

SEQ ID NO: 2 - protein, 254 aa, Escherichia coli
MRSIARRTAVGAALLLVMPVAVWISGWRWQPGEQSWLLKAAFWVTETVTQPWGVITHLILFGWFLWCLRFRIKAAFVLFAILAAAILVGQGVKSWIKDKVQEPRPFVIWLEKTHHIPVDEFYTLKRAERGNLVKEQLAEEKNIPQYLRSHWQKETGFAFPSGHTMFAASWALLAVGLLWPRRRTLTIAILLVWATGVMGSRLLLGMHWPRDLVVATLISWALVAVATWLAQRICGPLTPPAEENREIAQREQES